(19) United States
(12) Patent Application Publication
(10) Pub. No.: US 2026/0213012 A1
(43) Pub. Date: Jul. 23, 2026

(54) METHODS FOR EARLY-AGE EYE-TRACKING BIOMARKER FOR AUTISM SPECTRUM DISORDER

(71) Applicant: The Regents of the University of California, Oakland (CA)

(72) Inventors: Karen Pierce, San Diego, CA (US); Javad Zahiri, San Diego, CA (US)

(21) Appl. No.: 19/144,950

(22) PCT Filed: Jan. 3, 2024
(86) PCT No.: PCT/US2024/010185

§ 371 Date: Jun. 30, 2025

Related U.S. Application Data
(60) Provisional application No. 63/436,810, filed on Jan. 3, 2023.

(51) Int. Cl. G16H 50/20

(52) U.S. Cl. CPC G16H 50/20

(57) ABSTRACT

The disclosure relates to a method for determining if a child has autism, the method comprising: applying a trained machine learning model using eye tracking metrics from at least two eye tracking tests; and obtaining an autism risk score. The methods can further comprise obtaining the eye tracking metrics from the at least two eye tracking tests.

### CROSS-REFERENCE TO RELATED APPLICATIONS

This application claims the benefit of U.S. Provisional Appl. No. 63/436,810, filed Jan. 3, 2023, which is incorporated by reference as if fully set forth herein.

### GOVERNMENT SUPPORT CLAUSE

This invention was made with government support under MH118879 and MH080134 awarded by National Institutes of Health. The government has certain rights in the invention.

### BACKGROUND

Currently the only way to determine if a child has autism spectrum disorder (ASD) is to receive a developmental evaluation from an experienced clinician, such as a licensed clinical psychologist. There are often long waiting lists, and only a small number of clinicians have the experience required to make early diagnoses of ASD. Thus, there are many places in the country as well as worldwide wherein children wait months or years to receive a formal diagnosis due to a lack of available expertise. Moreover, diagnostic evaluations are expensive and usually cost the parent and/or insurance approximately $1,500-$2,500 per evaluation. Finally, clinical evaluations usually take between 2-3 hours to complete and result in fatigue for both the parent and toddler.

### SUMMARY

The method described herein for determining whether a child has ASD include an autism eye tracking test battery involving a ~15-minute eye tracking test battery designed to determine if a child has autism spectrum disorder (ASD) and has been validated for use between the ages of 12-48 months. The disclosure combines a toddler's eye tracking data with parent questions to arrive at an overall Autism Risk Score (ARS) or Autism Probability Score (APS), which terms may be used interchangeably herein. Autism risk can be calculated based on eye tracking alone, parent questions alone, and eye tracking+parent questions. The disclosure is based on the concept of a dynamic system wherein the precision and accuracy of the overall ARS continues to improve as new data from each child that takes the test is entered into the Autism Risk Score Master Datasheet.

### DESCRIPTION OF THE DRAWINGS

The drawings illustrate generally, by way of example, but not by way of limitation, various embodiments discussed herein.

FIG. 1 is a scheme showing the description of each of the 6 primary eye tracking tests (top) used in the methods described herein and the overall conceptualization of the Autism Probability Score (bottom). Briefly, a toddler participates in a maximum of 6 primary eye tracking tests that objectively quantify a toddler's visual social attention, gaze shifting, and auditory social attention. Additional information such as autonomic arousal and a parent's level of concern can be integrated into the model. Data sources that contribute to higher diagnostic classification accuracy are weighted more heavily in the final Autism Probability Score calculation.

FIG. 2 is a diagram showing a summary of the methods described herein.

FIG. 3 is a scheme of a machine learning engine for training and execution in accordance with the disclosure.

FIG. 4A is box plots illustrating the raw data for the GeoPref Test. TP=True positive; FP=False positive. Note: TP and FP designations are based on the 69% fixation on geometric images threshold. ASD=Autism Spectrum Disorder; GDD=Global Developmental Delay; LD=Language Delay, Other=Other delay such as a motor delay; TD=Typical development; TypSibASD=A typically developing toddler that has an ASD sibling.

FIG. 4B is a plot showing the effect sizes highlighting the comparison of eye tracking performance between different diagnostic groups during the GeoPref Test.

FIG. 4C is a plot showing diagnostic accuracy across different ages.

FIG. 5 is box plots illustrating % Fixation data from the 5 preferential looking paradigms and Gaze Shift data from the Joint Attention task. Data are stratified across 4 major diagnostic groups (age range 12-48 months, mean age 27 months). Because 5/6 tests are preferential looking paradigms, each toddler has both a social and non-social % fixation value that sum to 100%. For example, if a toddler fixates on geometric images 90% of the time, he/she has a corresponding value of 10% social fixation. Sample sizes and validation statistics using fixation levels associated with 97% specificity are included on the right side of each plot. Dashed line indicates threshold associated with 97% specificity for each test (except for Joint Attention, which is shown at 96% specificity), and green triangles indicate mean values. Cohen's d values are based on an ASD vs. TD contrast. TP=True Positive, FP-False Positive.

FIG. 6 is graphs illustrating the Accuracy (ACC), Negative Predictive Value (NPV), Positive Predictive Value (PPV), Specificity (SP) and Sensitivity (SN) of parent questions (crosshatched), eye tracking along (horizontal hatched) and eye tracking+parent questions combined (vertical hatched). As illustrated, eye tracking alone is highly accurate, but performance improves slightly with the inclusion of parent report questions.

FIG. 7 is a scheme showing a summary of the methods described herein where “GET SET EARLY” is a name ascribed to the methods described herein.

Unless otherwise indicated, all figures and drawings in this document are not to scale and are chosen for the purpose of illustrating different embodiments of the invention. The dimensions of the various components are depicted in illustrative terms only, and no relationship between the dimensions of the various components should be inferred from the drawings, unless so indicated. Although terms such as “top”, “bottom”, “upper”, “lower”, “under”, “over”, “front”, “back”, “up” and “down”, and “first” and “second” can be used in this disclosure, it should be understood that those terms are used in their relative sense only unless otherwise noted.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

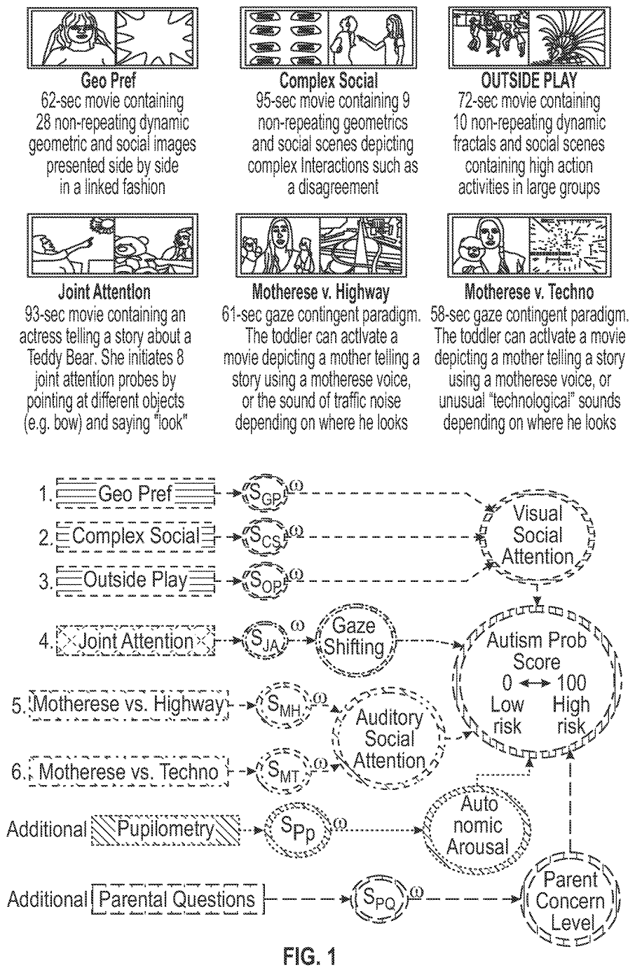

Autism is a heterogenous disorder with multiple biological systems and brain regions affected to varying degrees across children. In order to successfully detect the largest number of children possible, multiple eye tracking tests are utilized in the methods described herein that, in theory, may tap into a wide range of impacted neural systems since it is unlikely that a single test alone will be able to detect all children on the spectrum. The methods described herein for determining whether a child has ASD uses an eye tracking test battery (FIG. 1) that leverages six different eye tracking tests that are divided into five conceptual domains: (i) visual social attention, (ii) gave shifting; (iii) auditory social attention; (iv) autonomic arousal; and (v) parent concern level. The six eye tracking tests and one or more of the five conceptual domains may be supported by relatively distinct neural systems. Different metrics, such as percent fixation and number of saccades per second, are extracted from the dataset for each toddler on each test and combined to create an overall ARS/APS. The names of each eye tracking test can be found on the left, in FIG. 1, and a description of each test appears below each name. The eye tracking test battery and resulting ARS is based on a dynamic and flexible bioinformatics system wherein new eye tracking tests and/or metrics can be added at any time to existing tests to continually improve the accuracy of the ARS.

The methods for determining whether a child has ASD are superior to a formal diagnostic evaluation in at least the following ways.

First, the methods described herein are fast. The methods described herein can be completed in less than 30 minutes/and/or on a child that is less than 12 months old. For example, the eye tracking test battery described herein takes only ~15 minutes to complete, whereas a traditional evaluation can take anywhere from 2-3 hours. In stark contrast, one way for a clinician to diagnose autism is based on clinical judgement and diagnostic criteria from the Diagnostic and Statistical Manual 5th Edition and/or the Autism Diagnostic Observation Schedule, which is a standardized instrument designed to assist clinicians in diagnosing autism. It will usually take a clinician between 2-3 hours to make a clinical diagnosis of autism. But aside from a clinical diagnosis from a clinician, there are no existing medical tests for the early diagnosis of autism.

Second, the methods described herein can be fun for the child being evaluated. The test battery is more enjoyable for toddlers than a traditional diagnostic clinical evaluation. Specifically, during the eye tracking test battery, toddlers sit on their parent's lap, or in a car seat and watch a series of brief, child-friendly movies (e.g., a woman smiling and playing with a teddy bear). In contrast, during a traditional diagnostic evaluation, the clinician asks the child questions and requires him/her to do certain things that may be taxing to the child. Children often cry and tantrum during traditional diagnostic evaluations. While this can occur during eye tracking as well, the frequency is much lower, and children appear to enjoy it.

Third, the methods described herein can be objective. Unlike a traditional diagnostic evaluation which is based on a clinician's subjective interpretation of a child's behavior, the eye tracking battery generates quantitative results. Participants will receive a score associated with their performance on each of the individual eye tracking tests, as well as an overall Autism Risk Score ranging from 0 to 100 designating level of risk.

Fourth, the methods described herein are validated for use at very young ages. The most recent report notes that the mean age of ASD diagnosis in the U.S. about 4 years old. The methods described herein can be used at very young ages when it is often difficult to obtain an accurate diagnosis from clinicians. The methods described herein can identify a child with autism as young as 12 months in age. Furthermore, it is possible that this test battery can be used with infants at an even younger age, such as 9 months.

Fifth, because several of the eye tracking tests described herein have no sound at all, in theory, the methods described herein can be used with infants of any race, ethnicity, or language origin. Recent studies evaluating the GeoPref Test, which is one of the eye tracking tests in the battery, demonstrated comparable validity between ethnic groups, such as between Hispanic and non-Hispanic children.

And sixth, the methods described herein are precise. Unlike traditional diagnostic evaluations performed by a psychologist that examine children uniformly and do not take gender and sometimes not age into consideration, the methods described herein will generate a single Autism Risk Score that will be normed based on age, and gender.

In addition, the methods for determining whether a child has ASD are superior to other methods known in the art that use a single eye tracking test that tap into a single domain and use a single metric to evaluate dysfunction. See, e.g., Perochon et al., Nat Med 29:2489-2497 (2023). https://doi.org/10.1038/s41591-023-02574-3; and Jones et al., JAMA Netw. Open 6: e2330145 (2023). doi: 10.1001/jamanetworkopen.2023.30145, each of which is incorporated by reference as if fully set forth herein.

Briefly, the Perochon publication describes the use of screen for autism using iPads and, as a result, use the device's built-in camera to estimate where the child might be looking on a screen based on head position. But this method is an approximation of true eye tracking. The instant method uses an eye tracking device that leverages multiple cameras and measures actual point of gaze at anywhere from 120 to 600 HZ, though this is only one example of how the methods described herein can be implemented. Further, the Perochon publication merely screens to detect who might have autism and relies on an evaluation by a licensed clinical psychologist later. The methods described herein is more of a diagnostic tool, since it has very high diagnostic accuracy.

The Jones publication describes a subjective way to evaluate children for whether or not they have ASD by having a child view a single 8-to-10-minute video followed by analysis of gaze behavior across the entire single video. Jones bases diagnostic judgments on visual fixation and scanning, then determines whether or not a child deviates from what they consider normal. Jones, therefore, does not combine the various metrics used by the methods described herein.

The disclosure relates to, among other things, a method for determining if a child has autism, the method comprising:
obtaining eye tracking metrics (e.g., total looking time, percent fixation on non-social images, number of saccades per second, percent fixation on face, and number of joint attention alterations) from at least two (e.g., at least three, at least four, at least five or six eye tracking tests; two or three, two to four, three to four, two to five, three to five, two to six, three to six, four to six, five to six, or four to six) eye tracking tests;
applying a trained machine learning model using the eye tracking metrics; and
obtaining an autism risk score.

In another example, the disclosure relates to a method for determining if a child has autism, the method comprising:
applying a trained machine learning model using eye tracking metrics from at least two eye tracking tests; and
obtaining an autism risk score.

As shown in FIG. 1, the at least two eye tracking tests can be selected from the GeoPref test, the complex social test, the outside play test, the joint attention test, and a motherese test. Briefly, the GeoPref test uses a 62 second movie containing 28 non-repeating dynamic geometric and social images presented side-by-side in a linked fashion. The complex social test uses a 95-second movie containing 9 non-repeating geometrics and social scenes depicting complex interactions, such as a disagreement. The outside play test uses a 72-second movie containing 10 non-repeating dynamic fractals and social scenes containing high action activities in large groups. The joint attention test uses a 93 second movie containing an actress telling a story about a teddy bear. She initiates 8 joint attention probes by pointing at different objects (e.g., a bow) and saying “look.” The motherese test can either be the motherese vs. highway or the motherese vs. techno. The former is a 61-second gaze contingent paradigm where the child (e.g., a toddler) activates a movie depicting a mother telling a story using a “motherese” voice or the sound of traffic noise depending on where the child looks. The latter is a 58-second gaze contingent paradigm where the child (e.g., a toddler) activates a movie depicting a mother telling a story using a “motherese” voice or unusual “technological” sounds depending on where the child looks. The motherese vs. highway test and the motherese vs. techno test can each be used separately or in combination.

Those of skill in the art would recognize that each of the GeoPref test, the complex social test, the outside play test, the joint attention test, and a motherese test can be implemented in various other ways, not just the ways that are described herein. Further, those of skill in the art would recognize that each of the at least two eye tracking tests can be used in combination with other metrics such as metrics that measure autonomic nervous system arousal. Examples of such additional metrics include, but are not limited to, pupillometry, level of parent concern, and a combination thereof. Pupillometry relates to measuring the child's pupil size to establish a level of arousal. Parental questions, and the answers thereto, are used to establish a level of parent concern. For example, the answers to parental questions can elicit information about whether or not a parent has concerns about their child's development (yes/no) and/or information relating to visual and auditory attention (e.g., such as whether or not a child responds when his/her name is called). Questions can also relate to the presence or absence of known autism risk factors such as a premature birth or advanced maternal or paternal age.

Any of the at least two eye tracking tests (e.g., GeoPref test, the complex social test, the outside play test, the joint attention test, and a motherese test (includes motherese vs. traffic test and the motherese vs. techno test)) can be weighted so that tests that are more accurate will make a greater contribution to the autism risk score than those that are less accurate. These weights will be learned and tuned as a set of hyper parameters using the training and validation data sets.

The methods described herein can further comprise indexing/quantifying a child's visual social attention; indexing/quantifying a child's gaze-shifting; and/or indexing/quantifying a child's auditory social attention. Alternatively, or in addition, the methods described herein can further comprise weighting the indexing of a child's visual social attention; weighting the indexing a child's gaze-shifting; and/or weighting the indexing a child's auditory social attention.

Registering an autism risk score of 50 and lower indicates that the child would not meet the diagnostic criteria for ASD when a child is formally evaluated. Registering an autism risk score of 50 or higher or 51 or higher indicates that the child would meet the diagnostic criteria for autism spectrum disorder (ASD) when a child is formally evaluated (e.g., by a licensed clinical psychologist) and the higher the score the higher the autism probability and symptom severity. The diagnostic criteria for ASD can comprise persistent deficits in social communication and social interaction across multiple contexts. Persistent deficits in social communication and social interaction across multiple contexts can comprise deficits in social-emotional reciprocity; deficits in nonverbal communicative behaviors used for social interaction, and deficits in developing, maintaining, and understanding relationships. The diagnostic criteria for ASD can further comprise restricted, repetitive behaviors such as stereotyped or repetitive motor movements, use of objects, or speech; insistence on sameness, inflexible adherence to routines, or ritualized patterns of verbal or nonverbal behavior; highly restricted, fixated interests that are abnormal in intensity or focus; and/or hyper- or hyporeactivity to sensory input or unusual interest in sensory aspects of the environment.

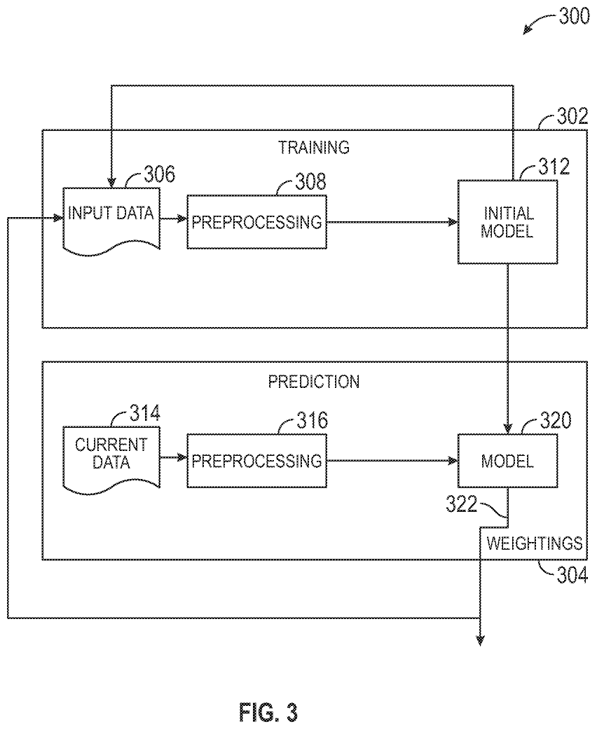

Making reference to FIG. 3, to generate an ARS/APS (Autism Risk Score/Autism Probability Score), a server (not shown) or software implemented on a processing circuitry (not shown) may store one or more machine learning models.

FIG. 3 illustrates a machine learning engine 300 for training and execution in accordance with the methods described herein. The machine learning engine 300 may be deployed to execute on a web server or as a stand-alone software.

Machine learning engine 300 uses a training engine 302 and a prediction engine 304. Training engine 302 uses eye tracking metrics 306 (e.g., total looking time, % fixation on non-social images, # of saccades per second, % fixation on face, # of joint attention alterations) to train, fine tune, and select the best performing classifier for each feature type combination. For each combination, all classifiers will be trained and fine-tuned using the training data. Then, according to the performance on the validation data, best performing models will be selected to deploy as the final set of models.

In the prediction engine 304, firstly, relevant features will be calculated from the raw eye tracking data that pass the quality control. Then, preprocessing pipeline will be applied on the features to normalize the feature, as discussed further below.

The training engine 302 may operate in an offline manner to train the model 320 (e.g., on a server). The prediction engine 304 may be designed to operate in an online manner (e.g., in real-time). In some examples, the model 320 may be periodically updated via additional training (e.g., via updated input data 306).

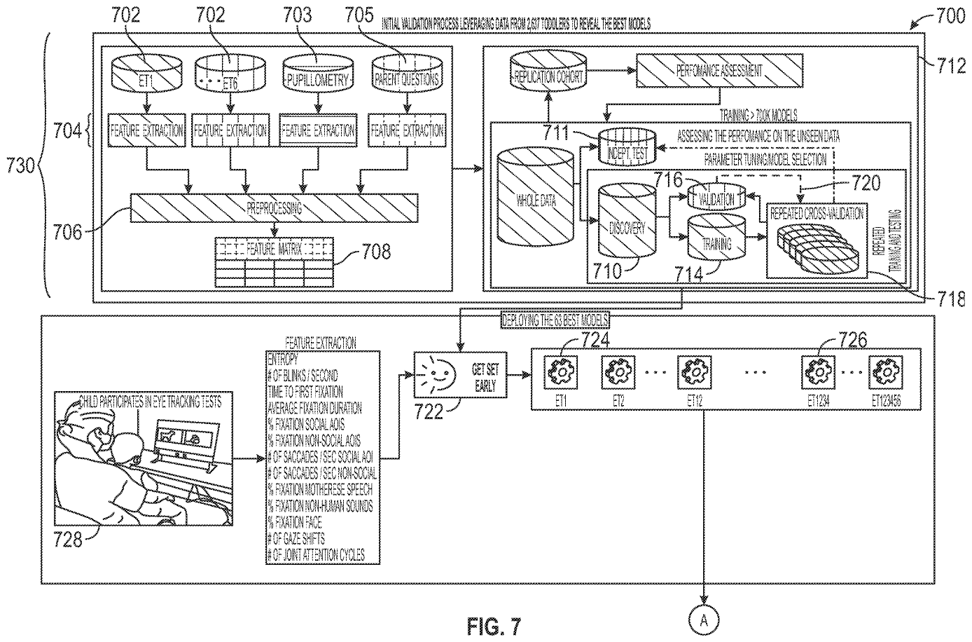

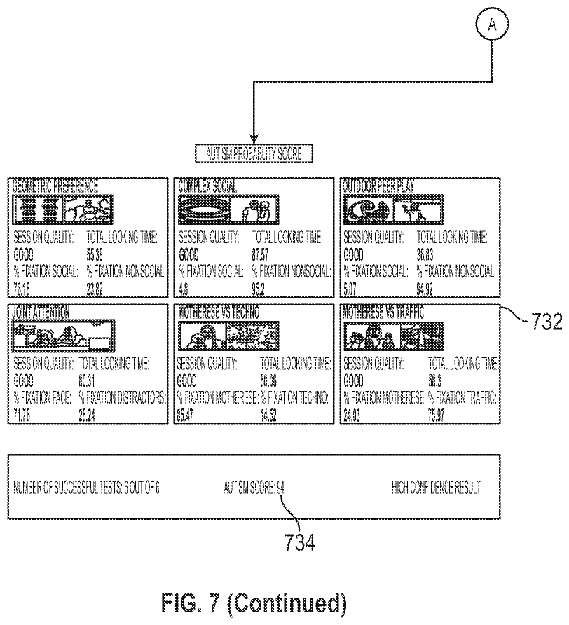

The initial model 312 may be updated using further input data 306 until a satisfactory model 320 is generated. The model 320 generation may be stopped according to a specified criteria when a satisfactory sensitivity, specificity, positive predictive value, negative predictive value, and/or area under the curve (see Table 1) is reached. Weighting 322 can be optionally applied. The model 320 can then generate an APS 324. FIG. 7 provides a more detailed overview of the claimed methods and is described below in greater detail.

The specific machine learning algorithm used for the training engine 302 may be selected from among many different potential supervised machine learning algorithms. Examples of supervised learning algorithms include artificial neural networks, Bayesian networks, support vector machines, decision trees (e.g., Iterative Dichotomiser 3, C9.5, Classification and Regression Tree (CART), Chi-squared Automatic Interaction Detector (CHAID)), random forests, linear discriminate analysis (LDA), quadratic discriminate analysis (QDA), k-nearest neighbor, linear regression, logistic regression, and XGBoost.

In some embodiments, the machine learning model used can be an ensemble of diverse base classifiers including Gradient Boosting, Quadratic Discriminant Analysis, Logistic Regression, Baysian network, Support Vector Machine (SVM), K Nearest Neighbor, Decision Tree, Random Forest, Random Tree, Naive Bayes Classifier, or Multi-Layer Perceptron (MLP).

Once trained, the model 320 can predict/determine whether a child has autism. The methods described herein can further comprise a validation step that can be used to, among other things, determine the best machine learning algorithm for each of 63 possible eye tracking combinations. The validation step can provide a sensitivity of at least about 50% (e.g., at least about 55%, at least about 60%, at least about 65%, at least about 70%, at least about 75%, at least about 80%, at least about 85%, at least about 90%, at least about 95% or higher; from about 50% to about 99%; about 50% to about 90%, about 60% to about 85%, about 50% to about 75%, about 60% to about 90% or about 55% to about 70%); a specificity of at least about 85% (e.g., at least about 90%, at least about 95%, at least about 96%, at least about 97%, at least about 98% or at least about 99%; from about 85% to about 99%; about 85% to about 90%, about 90% to about 99%, about 89% to about 93%, or about 95% to about 99%); a positive predictive value of at least about 80%; and/or a negative predictive value of at least about 60% (e.g., at least about 65%, at least about 70%, at least about 75%, at least about 80%, at least about 85%, at least about 90%, at least about 95% or higher; from about 60% to about 99%; about 60% to about 90%, about 60% to about 85%, about 65% to about 85%, about 70% to about 90% or about 80% to about 95%).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

Methods supporting diagnostic classification accuracy occurred across several stages. The results presented herein are based on eye tracking data for over 2,637 toddlers with and without autism. Given that toddlers with autism spectrum disorder may appear similar clinically to a toddler that has some other non-ASD delay (e.g., language delay), it was critical that the initial research, as illustrated here, contains toddlers from multiple non-ASD contrast groups. The non-ASD contrast groups contained toddlers with a language delay (LD), global developmental delay (DD), other delay (e.g., motor delay), typical development (TD), sibling of an ASD proband (Typ Sib), as well as those that show some autism characteristics but not enough to receive a formal ASD diagnosis (ASD Features). All subjects that participated in the studies received a diagnostic evaluation by a licensed clinical psychologist blind to eye tracking scores. The diagnosis provided by the licensed clinical psychologist is used as the ground truth determine the accuracy of eye tracking results and the APS. The overall process is described schematically in FIG. 7 where “GET SET EARLY” is a name ascribed to the methods described herein.

In FIG. 7, is an example of the claimed method 700 where raw data are extracted from eight different sources (e.g., six eye tracking (ET) tests 702, pupillometry test 703, and a set of parent questions 705). These data, after preprocessing 706, are used to train machine learning models 712 to predict autism and generate autism probability score 734. Raw data are collected for six eye tracking tests 702 (ET1: Geometric Preference; ET2: Complex Social; ET3: Outdoor Peer Play; ET4: Motherese vs. Traffic; ET5: Motherese vs. Techno; and ET6: Joint Attention). Additionally, pupillometry and parent questions are used as complementary information to boost prediction performance. Relevant features 704 are extracted from each of the eight data types including: number of blinks per second, time to the first fixation, average fixation duration, percent fixation social areas of interest (AOIs), percent fixation non-social AOIs, number of saccades per second social AOIs, number of saccades per second non-social AOIs, percent fixation motherese speech, percent fixation non-human sounds, percent fixation face, number of gaze shifts, number of joint attention cycles, gestational age, gender, and parent concern. Poor quality data is removed and then features are normalized in a preprocessing step 706. Each subject's data is represented as a numerical feature vector and whole data is converted to a feature matrix 708 where each row corresponds to a toddler and each column represents a feature. The obtained feature matrix 708 is divided into a discovery (80%) dataset 710 and independent test set (20%) 711. The discovery dataset 710 is also divided into training (80%) 714 and validation set (20%) 716. In one example, repeated five-fold cross-validation 718 is used to train and to perform parameters fine tuning 720. According to the cross-validation performance, the best model is selected and assessed on the validation set and parameter tuning and model selection is repeated if needed. Then the best performing models are selected and evaluated on the replication dataset which were collected independently to assess the generalization performance. Based on the performance, if needed, training and validation are repeated. Finally, the best model for each 63 eye-tracking combination is selected to deploy as the “GET SET EARLY” software application 722. For example, “ET1” 724 can be the best model for those kids who participated only in ET1, which is Geometric Preference test, or “ET1234” 726 represents the best model for those kids who participated only in ET1, ET2, ET3, and ET4. In a clinic 728 a toddler may take some or all of the provided tests (e.g., six eye tracking tests). The obtained raw data are extracted, preprocessed, and using the same steps described in steps 730. The extracted data is fed to the “GET SET EARLY” software application as a numerical feature vector. According to the number successful test that the toddler takes, the corresponding trained machine learning model is selected and an autism probability score 734. The “GET SET EARLY” software application generates a report 732 for all successful tests and autism probability score 734. An autism probability score 734 greater than 50 (e.g., 50 or higher or 51 or higher) is interpreted as autism and the higher the score the higher the autism probability and symptom severity.

### Stage 1: Thorough Examination of Each Test Individually

The first stage was designed to estimate classification performance for each of our eye tracking tests individually using various metrics including: sensitivity, specificity, positive predictive value (PPV), negative predictive value (NPV), and Area Under the ROC Curve (AUC-ROC). See, e.g., Pierce et al., Archives of General Psychiatry 68: 101-109 (2011). https://doi.org:10.1001/archgenpsychiatry.2010.113; Pierce et al., Biological Psychiatry 79: 657-666 (2016). https://doi.org:10.1016/j.biopsych.2015.03.032; Moore et al., Molecular Autism 9: 1-13 (2018). https://doi.org:10.1186/s13229-018-0202-z; Wen et al., Sci. Rep. 12:4253 (2022). https://doi.org:10.1038/s41598-022-08102-6; and Pierce et al., JAMA Netw. Open 6, e2255125 (2023). https://doi.org:10.1001/jamanetworkopen.2022.55125, each of which is incorporated by reference as if fully set forth herein. To do so, toddlers were designated as ASD or non-ASD (e.g., ASD Features, Delay, Other delay, Typ Sib and TD) based on their most recent diagnosis received by a licensed clinical psychologist blind to eye tracking scores. A receiver operating curve (ROC) was generated for each test to determine the optimal percent fixation and/or saccades per second levels associated with specificity >96% for each eye tracking test. A high specificity rate was chosen given expense and anxiety associated with a false positive result. Toddlers were then designated as either a true positive (TP) case (e.g., a toddler with ASD whose percent fixation score exceeded the predetermined threshold), a false positive (FP) case (e.g., a non-ASD toddler whose percent fixation score exceeded a predetermined threshold), a true negative (TN) case (e.g., a non-ASD toddler whose fixation score did not exceed the predetermined threshold) or a false negative (FN) case (e.g., a toddler with ASD whose fixation score did not exceed the predetermined threshold). A standard 2×2 contingency table for binary classification was used to calculate metrics (e.g., sensitivity).

### Stage 2: Validation—Determining the Best Machine Learning Algorithm for Each of 63 Possible Eye Tracking Combinations

The second stage was designed to determine the optimal process for combining data across multiple eye tracking tests to generate an accurate APS. Multiple eye tracking features (e.g., total looking time, % fixation on non-social images, # of saccades per second, % fixation on face, # of joint attention alterations) across each of the existing six (6) eye tracking tests were leveraged and compared across a range of diverse classifiers such as Linear Discriminant Analysis (LDA), Quadratic Discriminant Analysis (QDA), regression methods (e.g. logistic regression, lasso, elastic net), Support Vector Machine (SVM), Sequential Miminimal Optimization (SMO), K Nearest Neighbors, Decision Tree methods (e.g. J-48, C4.5, CART, XGBoost), Random Forest, Naive Bayes Classifier, Baysian network, Adaboost and Multi-Layer Perceptron (MLP). An expanded range of metrics were considered including not only sensitivity, specificity, PPV and NPV, but also accuracy, F-1 score, and Matthew correlation coefficient (MCC) to determine which model was performing better. Grid search was used, and a five-fold cross-validation procedure was repeated to tune the hyper parameters for each classifier. Then, based on the performance of the optimized classifiers on the held-out validation set, the best performing classifiers were selected. Finally, using the independent test set the performance of the final APS was estimated on the unseen data. Based on the validation and test performance, the trained machine learning models (also referred to herein as “bioinformatics model”) was updated and retrained as described herein. The data are shown in Table 1 herein. Table 1 shows the classification accuracy for ASD diagnosis based on results from illustrative training and independent hold test dataset. Numbers denoted in the Eye Tracking Test combination are based on the numbers shown in FIG. 1 (1=Geo Pref Test; 2=Complex Social, and so on). Note that the Independent Test Set was not used during training. Results from this dataset reflects how well the results generalized to the general population. ML=Machine Learning.

TABLE 1

| | | | | | | | Best |
|---|---|---|---|---|---|---|---|
| Eye Tracking Test | | | | | | | Performing ML |
| Combination | N | Sensitivity | Specificity | PPV | NPV | AUC | Model |
| 123456-Training | 86 | 1.00 | 1.00 | 1.00 | 1.00 | 1.00 | Adaboost |
| 123456- Independent | 22 | 0.92 | 0.80 | 0.85 | 0.89 | 0.86 | |
| Test | | | | | | | |
| 1234 - Training | 141 | 1.00 | 1.00 | 1.00 | 1.00 | 1.00 | Gradient |
| 1234-Independent | 36 | 0.78 | 0.85 | 0.90 | 0.69 | 0.78 | Boosting |
| Test | | | | | | | |
| 1235-Training | 137 | 0.83 | 0.95 | 0.96 | 0.79 | 0.83 | Quadratic |
| 1235-Independent | 35 | 0.73 | 0.85 | 0.89 | 0.65 | 0.73 | Discriminant |
| Test | | | | | | | Analysis |
| 1245- Training | 89 | 0.86 | 0.97 | 0.98 | 0.80 | 0.96 | Logistic |
| 1245- Independent | 23 | 0.76 | 0.83 | 0.93 | 0.56 | 0.78 | Regression |
| Test | | | | | | | |

### Stage 3: Proprietary Coding and Software

During the third stage, based on the validation results obtained during Stage 2, the final pipeline was implemented to generate an APS and to create software. First, the software determines the number of eye tracking tests, parent questions and other information validly collected for each child. Second, the software matches the number of eye tracking tests completed and other available data with the trained machine learning model that has been determined during the validation stage to provide the most accurate APS. Third, the software generates an Autism Probability Score by applying the model on the extracted features. Fourth, the software generates a pdf summarizing the child's performance on each of the 6 primary tests (or whatever combination the toddler successfully completed) as well as the APS.

### Stage 4: Optimizing End-User Experience and Ease of Use

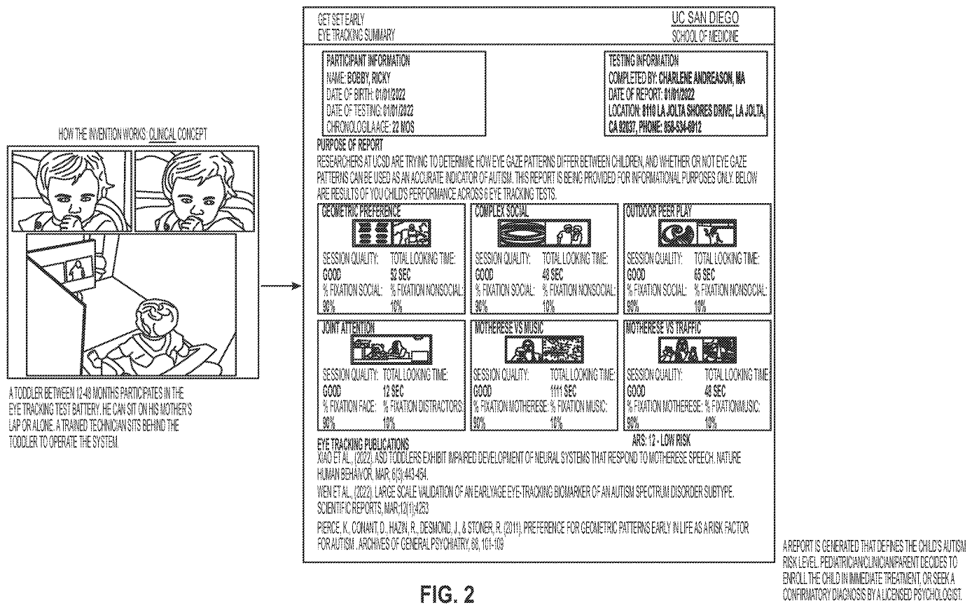

The fourth stage was designed to test usability of the system by making sure the software is easy to execute using a user-friendly GUI and that the diagnostic report (see FIG. 2) is rapidly generated following conclusion of the eye tracking tests.

### Data and Findings

Results from a Single Eye Tracking Test, the GeoPref Test, Using % Fixation on Geometric Images (N=1,863 Toddlers)

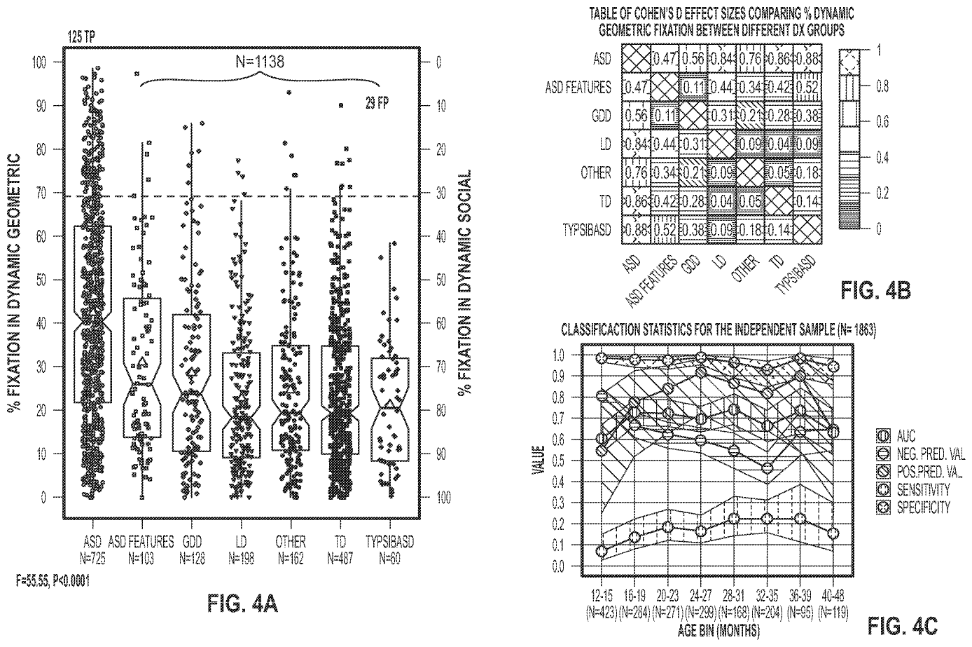

In a cohort of 1,863 toddlers spanning 7 different diagnostic categories (ASD, ASD Features, Global Developmental Delay (GDD), Language Delay (LD), “Other” delay, Typical Development (TD) and Typical Sibling of an ASD proband (TypSibASD), there were only 29 false positives associated with the GeoPref Test. See FIG. 4A. This translates into a 2% false positive rate and an overall specificity rate of 98%. This was calculated using the 69% fixation threshold on geometric images for the GeoPref Test (FIG. 4A). Sensitivity was modest at 17% underscoring the heterogeneity inherent in ASD and the fact that the GeoPref test is capable of detecting a unique subtype within the ASD spectrum. This also shows the considerable validation work that has been put into this test. See Wen et al., Sci Rep 12:4253 (2022). https://doi.org:10.1038/s41598-022-08102-6, which is incorporated by reference as if fully set forth herein. FIG. 4B shows the effect sizes highlighting the comparison of eye tracking performance between different diagnostic groups during the GeoPref Test. FIG. 4C shows diagnostic accuracy across different ages. TP=True positive; FP=False positive. Note: TP and FP designations are based on the 69% fixation on geometric images threshold. ASD=Autism Spectrum Disorder; GDD=Global Developmental Delay; LD=Language Delay, Other=Other delay such as a motor delay; TD=Typical development; TypSibASD=A typically developing toddler that has an ASD sibling.

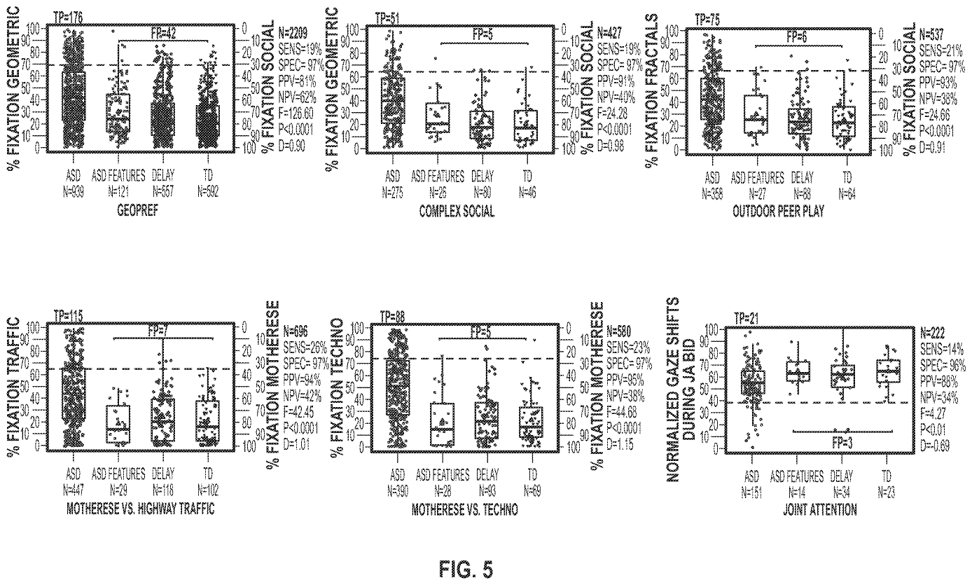

Making reference to FIG. 5, the figure is boxplots illustrating % Fixation data from the 5 preferential looking paradigms and Gaze Shift data from the Joint Attention task. Data are stratified across 4 major diagnostic groups (age range 12-48 months, mean age 27 months). Because 5/6 tests are preferential looking paradigms, each toddler has both a social and non-social % fixation value that sum to 100%. For example, if a toddler fixates on geometric images 90% of the time, he/she has a corresponding value of 10% social fixation. Sample sizes and validation statistics using fixation levels associated with 97% specificity are included on the right side of each plot. Dashed line indicates threshold associated with 97% specificity for each test (except for Joint Attention, which is shown at 96% specificity), and green triangles indicate mean values. Cohen's d values are based on an ASD vs. TD contrast. TP=True Positive, FP-False Positive.

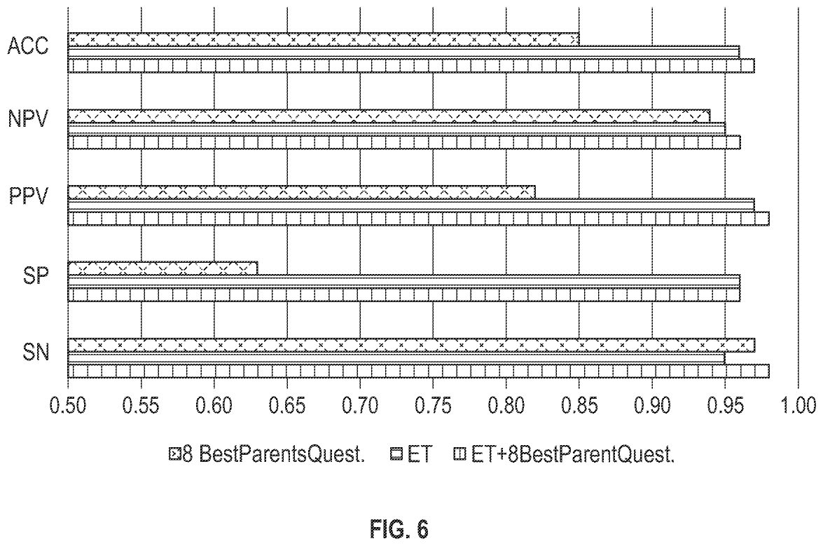

To reveal the diagnostic classification accuracy of a full 6-test battery with all metrics, and to examine potential synergy between eye tracking and parent questions (e.g., “When you call your child's name, does he respond?”) with parent questionnaire data alone (N=1,230 sex and age-matched toddlers; N=615 ASD, 615 Non-ASD), toddlers with relevant eye tracking data (e.g., % fixation on non-social AOIs; # of saccades within social and on-social AOIs and # of gaze alterations) from all 6 eye tracking tests (N=120; N=65ASD, 55=Non-ASD), and toddlers with data from all 6 eye tracking tests+parent questionnaire data (N=65; 41ASD, 24 Non-ASD) participated in a small pilot study. Using a decision tree (J48) classifier (see Zahiri et al., Curr. Genomics 14:397-414 (2013) https://doi.org:10.2174/1389202911314060004; and Goto et al., JAMA Netw. Open 2: e186937 (2019) https://doi.org:10.1001/jamanetworkopen.2018.6937), revealed that the parent questionnaire had only moderate diagnostic classification accuracy (FIG. 5). Between subjects t-tests, revealed that 8 parent questions (e.g., “When you call your child's name, does he respond by looking at you?”) were significantly different (p<0.05) between ASD and non-ASD toddlers. Eye tracking alone (“ET”; FIG. 6, horizontal hatched), however, demonstrated excellent classification accuracy, which was slightly improved with the addition of, for example, the 8 Communication and Symbolic Behavior Scales (CSBS) questions (FIG. 6 vertical hatched). See, e.g., Weatherby and Prizant (2003) Communication and Symbolic Behavior Scales, Normed Edition (CSBS) https://doi.org/10.1037/t11527-000.

### Generating an Autism Probability Score (APS)

The final classifier for calculating the APS is an ensemble learning approach. A grid search strategy was exploited to optimize the classifiers' parameters for a set of >20 classification algorithms. As the first step, the best classifiers were selected among >200k trained models based on the performance on the validation data. Then, the selected trained classifiers were calibrated to generate the optimum APS scores. These calibrated classifiers were used as the base classifiers in the ensemble learning model. As the next step, the ensemble model was trained via a grid search to optimize the generated APS using “negative logarithmic loss” as the objective function. The validation dataset was used for optimizing the APS. Finally, the performance of the APS on the unseen data was evaluated using the independent test set.

1. A method for determining if a child has autism, the method comprising:
obtaining eye tracking metrics from at least two eye tracking tests;
applying a trained machine learning model using the eye tracking metrics; and
obtaining an autism risk score.

2. The method of claim 1, wherein the trained machine learning model is matched to the number and combination of eye tracking tests that the child took.

3. The method of claim 1, wherein the at least two eye tracking tests are selected from the GeoPref test, the complex social test, the outside play test, the joint attention test, and a motherese tests.

4. (canceled)

5. The method of claim 1, further comprising adding metrics that measure autonomic nervous system arousal and/or further comprising a validation step.

6. The method of claim 5, wherein the metrics that measure autonomic nervous system arousal comprise pupillometry, level of parent concern, and a combination thereof.

7. (canceled)

8. The method of claim 5, wherein the validation step provides a sensitivity of at least about 50%; a specificity of at least about 85%; a positive predictive value of at least about 80%; and/or a negative predictive value of at least about 60%.

9. The method of claim 1, further comprising weighting each of the at least two eye tracking tests so that tests that are more accurate will make a greater contribution to the autism risk score than those that are less accurate.

10. The method of claim 1, further comprising indexing a child's visual social attention; further comprising indexing a child's gaze-shifting; and/or further comprising indexing a child's auditory social attention.

11. (canceled)

12. (canceled)

13. The method of claim 10, further comprising weighting the indexing of the child's visual social attention, the child's gaze-shifting, and/or the child's auditory social attention.

14. The method of claim 1, wherein autism risk score of 50 or higher indicates that the child would meet the diagnostic criteria for autism spectrum disorder (ASD) when a child is formally evaluated.

15. The method of claim 14, wherein the diagnostic criteria for ASD comprise persistent deficits in social communication and social interaction across multiple contexts.

16. The method of claim 15, wherein the persistent deficits in social communication and social interaction across multiple contexts comprise deficits in social-emotional reciprocity; deficits in nonverbal communicative behaviors used for social interaction, and deficits in developing, maintaining, and understanding relationships.

17. The method of claim 14, wherein the diagnostic criteria for ASD further comprises restricted, repetitive behaviors.

18. The method of claim 17, wherein the restricted, repetitive behaviors comprise stereotyped or repetitive motor movements, use of objects, or speech; insistence on sameness, inflexible adherence to routines, or ritualized patterns of verbal or nonverbal behavior; highly restricted, fixated interests that are abnormal in intensity or focus; and/or hyper- or hyporeactivity to sensory input or unusual interest in sensory aspects of the environment.

19. The method of claim 1, wherein the obtaining eye tracking metrics is from at least five eye tracking tests.

20. The method of claim 1, wherein trained machine learning model is selected from XGBoost, linear regression, linear discriminant analysis, and gradient boosting.

21. The method of claim 1, wherein the method can be/is completed in less than 30 minutes.

22. The method of claim 1, wherein the method can be/is performed on a child that is less than 12 months old.

23. The method of claim 1, further comprising treating the child using naturalistic developmental behavioral interventions (NDBI).

24. (canceled)

25. A method for determining if a child has autism, the method comprising:
applying a trained machine learning model using eye tracking metrics from at least two eye tracking tests; and
obtaining an autism risk score.
